(19) United States
(12) Patent Application Publication
(10) Pub. No.: US 2021/0177914 A1
(43) Pub. Date: Jun. 17, 2021

(54) CANNABINOID ENRICHED COMPOSITION AND METHOD OF USING

(71) Applicant: Cannabis Global, Inc., Los Angeles, CA (US)

(72) Inventor: Joseph Noel, Seal Beach, CA (US)

(21) Appl. No.: 17/120,042

(22) Filed: Dec. 11, 2020

Related U.S. Application Data
(60) Provisional application No. 62/946,894, filed on Dec. 11, 2019.

(51) Int. Cl. A61K 35/644; A61K 47/36; A61K 31/352; A61K 31/05; A61K 47/54; A61K 47/64

(52) U.S. Cl. CPC A61K 35/644; A61K 47/36; A61K 47/64; A61K 31/05; A61K 47/549; A61K 31/352

(57) ABSTRACT

A method of treating a medical condition addressed by one or more cannabinoids, comprising providing the cannabinoid enriched solid composition to a recipient in an amount sufficient to treat the medical condition. A composition and a method to produce the composition are also disclosed.

### CROSS REFERENCE TO RELATED APPLICATIONS

This application claims the benefit of and priority to provisional patent application U.S. 62/946,894, filed Dec. 11, 2019, the contents of which are fully incorporated by reference herein.

### BACKGROUND

The instant disclosure relates to a solid composition comprising one or more cannabinoids, and method of using this composition. In particular, wherein the cannabinoid composition is a dry free-flowing solid. Delivery of biologically active compounds such as pharmaceuticals and so-called nutraceuticals to living systems is the object of much study and research. Making a material available to a living system may be further complicated by the solubility profile of the material. While technologies exist, which may render a material with low water solubility available to a living system, such technologies typically have other drawbacks rendering their use limited to particular instances.

One attractive means of delivering biologically active material to living organisms, such as human beings, is via an ingestible carrier. Edible or otherwise consumable films may be adapted to be water and/or mucosally dissolvable and then swallowed by an end user. US 2004/0247647 is generally directed to a breath freshening film adapted to rapidly dissolve in the mouth of a consumer comprising a high viscosity and a low viscosity film forming agent such as hydroxypropyl methylcellulose (HPMC) for improved strength during processing and storage. Other references include US20170127711, which is generally directed to a water soluble package comprising HPMC, along with other materials to render a hydrophobic component ingestible.

U.S. Pat. No. 4,504,516A is directed to a method for preparing powdered honey products, to the products obtained by the method and to the use of the powdered bee products. The method for the preparation of a powdered honey product which contains at least 50 weight percent honey comprising the step of combining with agitation 5 to 25 weight percent pyrogenic or precipitated fine grained silica with 50 to 85 weight percent of a honey component which is honey or a mixture of honey and at least one bee product selected from the group consisting of beebread, pollen, royal jelly, drone syrup, queen syrup, beeswax, propolis and propolis extract at a temperature not exceeding 35° C., said percentages based on the final powdered honey product.

WO2005053431A1 is directed to a method for producing honey powder having granular, powdered or other various shapes by drying a composition in which crude honey and other additives are mixed, and a honey powder produced by the method. The method includes (a) preparing constituent ingredients by weighting 5 to 95 wt % of crude honey and 95 to 5 wt % of predetermined additive powder; (b) homogeneously mixing the prepared constituent ingredients; and (c) dry-powdering the mixed composition by removing moisture in the mixed composition using a predetermined drying manner. While crude honey was in a liquid state that is not easily dealt and stored and has a limit in development of products in various ways, this method solves such problems. Thus, the method will contribute to more profits of apicultural farmers as well as promote health of the people and development of relative industries by means of various kinds of honey products.

WO2008072998A1 is directed to a method for producing dry powder honey consists in heating, cooling, grinding and packaging in sealed containers, wherein heat treatment is carried out by means of microwave irradiation in vacuum at a temperature ranging from 60 to 110° C. and moisture of 5-7% in such a way that the product is hard-boiled, and an anti-balling agent is added during grinding. Aspects of this disclosure make it possible to increase the process performance and to produce the high quality dry powder honey used for the extended list of honey products and confectionery.

Numerous disclosures are directed to dry, flowable powders are known. However, they have low loading rates and thus perform poorly, often creating a sticky paste upon honey integration, versus the desirable dry, flowable powder.

### SUMMARY

In one aspect of the disclosure, a method of treating a medical condition addressed by one or more cannabinoids, comprises the steps of providing a feed mixture comprising one or more cannabinoids to an animal capable of producing a foodstuff; collecting a cannabinoid enriched foodstuff produced by or derived from the animal; combining a portion of the cannabinoid enriched foodstuff with an amount of an absorbent, an adsorbent, or a combination thereof, to produce a free-flowing cannabinoid enriched solid composition, and providing the cannabinoid enriched solid composition to a recipient in an amount sufficient to treat the medical condition.

In one aspect of the disclosure, a cannabinoid enriched composition comprises a cannabinoid enriched foodstuff produced by, or comprising a portion of an organism in the animal kingdom which has been fed with a feed comprising one or more cannabinoids.

In one aspect of the disclosure, a cannabinoid enriched composition comprises a cannabinoid enriched foodstuff produced by a living organism in the animal kingdom; in combination with an absorbent, an adsorbent, or a combination thereof, present in an amount such that the composition is a free-flowing solid.

### BRIEF DESCRIPTION OF THE DRAWINGS

FIG. 1 is a chromatogram showing the concentration of cannabinoids in a composition according to an embodiment of the disclosure;

FIG. 2 is a chromatogram showing the concentration of cannabinoids in a composition according to an embodiment of the disclosure;

FIG. 3 is a chromatogram showing the concentration of cannabinoids in a composition according to an embodiment of the disclosure; and

FIG. 4 is a chromatogram showing the concentration of cannabinoids in a composition according to an embodiment of the disclosure.

### DETAILED DESCRIPTION

For the various aspects of this disclosure and the claims thereto, the new numbering scheme for the Periodic Table Groups is used as described in CHEMICAL AND ENGINEERING NEWS, 63(5), p. 27 (1985). Therefore, a “Group 4 metal” is an element from Group 4 of the Periodic Table.

For purposes of this disclosure and claims thereto, the term “substituted” means that a hydrogen group has been replaced with a hydrocarbyl group, a heteroatom, or a heteroatom containing group. For example, a “substituted hydrocarbyl” is a radical made of carbon and hydrogen where at least one hydrogen is replaced by an alkyl group, a heteroatom or heteroatom containing group. The terms “hydrocarbyl radical,” “hydrocarbyl group,” or “hydrocarbyl” may be used interchangeably and are defined to mean a group consisting of hydrogen and carbon atoms only. Preferred hydrocarbyls are C₁-C₁₀₀ radicals that may be linear, branched, or cyclic, and when cyclic, aromatic or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl naphthyl, and the like.

Unless otherwise indicated, (e.g., the definition of “substituted hydrocarbyl”, “substituted cannabinol,” etc.), the term “substituted” means that at least one hydrogen atom has been replaced with at least one non-hydrogen group, such as a hydrocarbyl group, a heteroatom, or a heteroatom containing group, or at least one functional group such as a halogen (e.g., Br, Cl, F, I), —NR*₂, —NR*—CO—R*, —OR*, *—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

The term “substituted hydrocarbyl” means a hydrocarbyl radical in which at least one hydrogen atom of the hydrocarbyl radical has been substituted with at least one heteroatom (such as halogen, e.g., Br, Cl, F or I), —NR*₂, —NR*—CO—R*, —OR*, *—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

Other examples of functional groups include those typically referred to as amines, imides, amides, ethers, alcohols (hydroxides), sulfides, sulfates, phosphides, halides, phosphonates, alkoxides, esters, carboxylates, aldehydes, and the like.

Unless otherwise indicated, room temperature is 23° C. “Different” or “not the same” as used to refer to R groups in any formula herein (e.g., R² and R⁸ or R⁴ and R¹⁰) or any substituent herein indicates that the groups or substituents differ from each other by at least one atom or are different isomers.

Unless otherwise noted, all molecular weights are reported in units of g/mol or Daltons (Da). The following abbreviations may be used herein: Me is methyl, Et is ethyl, Pr is propyl, cPr is cyclopropyl, nPr is n-propyl, iPr is isopropyl, Bu is butyl, nBu is normal butyl, iBu is isobutyl, sBu is sec-butyl, tBu is tert-butyl, Oct is octyl, Ph is phenyl, Bn is benzyl, CBD refers to cannabidiol, THC refers to tetrahydrocannabinol, TPGS refers to tocopheryl polyalkylene glycol succinates and derivatives thereof, HPMC refers to hydroxypropyl methylcellulose and derivatives thereof, and the like.

For purposes herein, the terms “group,” “radical,” and “substituent” may be used interchangeably. A multivalent radical refers to a radical having two or more attachment points, e.g., methylene —CH₂— is a multivalent radical of methane.

Unless indicated otherwise, as used herein, a water soluble composition is defined as a composition in which 400 mg of the composition dissolves, (i.e., forms a clear solution) in 240 ml of water at a temperature of 20° C. with stirring within 30 seconds.

Unless indicated otherwise, as used herein, a water miscible composition is defined as a composition in which 400 mg of the composition disperses (i.e., forms a clear to turbid solution) in 240 ml of water at a temperature of 20° C. with stirring within 30 seconds, and in which at least 95 wt % of the composition remains dispersed in the mixture after 5 minutes without stirring.

As used herein, “colloid” refers to a mixture containing two phases, a dispersed phase and a continuous phase, with the dispersed phase containing particles (droplets) distributed throughout the continuous phase. Colloidal mixtures include aerosols, foams, and dispersions.

For purposes herein, a free-flowing solid, which may include a powder, refers to a solid composition in which the particles are not cohesive and do not cling to one another. Dry sand is an example of a free-flowing solid. Accordingly, free flowing powders have a positive angle of repose.

As used herein, discrete particles refer to individual domains comprising the cannabinoids and the absorbent carrier. The particle size refers to the average particle size, which may be calculated by various methods readily known in the art. Suitable methods for determining the average particle size include examination of either an SEM or AFM micrograph or image in which the number average particle size may be determined. For purposes herein, the size of any one particle is always determined along the longest axis of the particle. Accordingly, for purposes herein, the terms “particle size”, “average particle size”, “average maximum dimension” and the like are used interchangeably. Alternatively, information about particles in embodiments of the cannabinoid enriched solid composition or the mixture produced by dilution thereof can be expressed in terms of particle density, for example, ppm (parts per million), or percent solids, in the compositions.

As used herein, “surfactant” refers to synthetic and naturally occurring amphiphilic molecules that have hydrophobic portion(s) and hydrophilic portion(s). A “surfactant system” refers to combinations and/or blends or mixtures of surfactants to produce an intended characteristic. Examples of surfactant systems include so-called “matched pairs” of surfactants having different hydrophobe/lipophobe balance (HLB) characteristics.

As used herein, “HLB” refers to a value that is used to index and describe a surfactant according to its relative hydrophobicity/hydrophilicity, relative to other surfactants. A surfactant's HLB value is an indication of the molecular balance of the hydrophobic and hydrophilic portions of the surfactant, which is an amphipathic molecule. Each surfactant and mixture of surfactants (and/or co-surfactants) has an HLB value that is a numerical representation of the relative weight percent of hydrophobic and hydrophilic portions of the surfactant molecule(s). HLB values are derived from a semi-empirical formula. The relative weight percentages of the hydrophobic and hydrophilic groups are indicative of surfactant properties, including the molecular structure, for example, the types of aggregates the surfactants form and the solubility of the surfactant. See, for example, Griffin (1949) J. Soc. Cos. Chem. 1:311. Surfactant HLB values range from 1-45, while the range for non-ionic surfactants typically is from 1-20. The more lipophilic a surfactant is, the lower its HLB value. Conversely, the more hydrophilic a surfactant is, the higher its HLB value.

Due to their amphiphilic (amphipathic) nature, surfactants typically can reduce the surface tension between two immiscible liquids, for example, the oil and water phases in an emulsion, stabilizing the emulsion. Surfactants may be characterized herein based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10.

As used herein, “co-surfactant” is used to refer to a surfactant that is used in the provided compositions in combination with the primary surfactant, for example, the cannabinoid enriched solid composition described herein, for example, to improve the emulsification of the provided compositions and/or compounds, for example, to emulsify the ingredients upon dilution. In one example, the provided compositions can contain at least one surfactant and at least one co-surfactant. Typically, the co-surfactant represents a lower percent, by weight of the provided compositions, compared to the surfactant. Thus, the provided compositions typically have a lower concentration of the co-surfactant(s) than of the surfactant.

As used herein, “micelle” refers to aggregates formed by surfactants that typically form when a surfactant is present in an aqueous composition, typically when the surfactant is used at a concentration above the critical micelle concentration (CMC). In micelles, the hydrophilic portions of the surfactant molecules contact the aqueous or the water phase, while the hydrophobic portions form the core of the micelle, which can encapsulate the non-polar cannabinoids and other ingredient(s). Typically, the surfactants form micelles containing the cannabinoids within either as the particles are formed, upon dilution of embodiments of the cannabinoid enriched solid composition or the mixture produced by dilution thereof in water, or both. Typically, the micelles in embodiments of the cannabinoid enriched solid composition or the mixture produced by dilution thereof have an average particle size of less than or equal to about 1000 nm, typically less than or less than about 500 nm, typically less than 300 or less than about 300 nm, for example, less than 250 nm or less than about 250 nm, for example, less than 200 nm or less than about 200 nm, for example, less than or less than about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm.

As used herein, “analog” and/or “derivative” refers to a chemical compound that is structurally similar to another compound (referred to as a parent compound), but differs slightly in composition, for example, due to the variation, addition or removal of an atom, one or more units (e.g., methylene units, —(CH₂)ₙ—) or one or more functional groups, e.g., a glycoside of a phenolic compound is a phenolic ether analog and/or derivative of the phenolic parent compound. The analog and/or derivative can have different chemical or physical properties compared with the original compound and/or can have improved biological and/or chemical activity. Alternatively, the analog and/or derivative can have similar or identical chemical or physical properties compared with the original compound and/or can have similar or identical biological and/or chemical activity. For example, the analog and/or derivative can be more hydrophilic, or it can have altered reactivity as compared to the parent compound. The analog and/or derivative can mimic the chemical and/or biological activity of the parent compound (i.e., it can have similar or identical activity), or, in some cases, can have increased or decreased activity. The analog and/or derivative can be a naturally or non-naturally occurring (e.g., synthetic) variant of the original compound. Other types of analogs and/or derivatives include isomers (e.g., enantiomers, diastereomers) and other types of chiral variants of a compound, as well as structural isomers. The analog and/or derivative can be a branched or cyclic variant of a linear compound. For example, a linear compound can have an analog and/or derivative that is branched or otherwise substituted, e.g., a saccharide, to impart certain advantageous properties (e.g., improved hydrophobicity or bioavailability).

As used herein, “organoleptic properties” refers to sensory attributes of a food or beverage, in particular upon dilution of the cannabinoid enriched solid composition into a beverage. Those of skill in the art understand such properties and they can be quantitated if needed. Organoleptic properties include, but are not limited to, taste, odor and/or appearance. “Desirable” or “advantageous” organoleptic properties include those organoleptic properties of a food or beverage composition for consumption by an average human subject, such as a desirable odor, taste and/or appearance, or the lack of an undesirable odor, taste and/or appearance. Undesirable organoleptic properties include the presence of, for example, an undesirable taste, odor or appearance attribute, such as the presence of an “off-taste” or “off-odor,” for example a fishy, grassy, metal or iron, sharp or tingling taste or odor, or the presence of an undesirable appearance attribute, such as separation or precipitation. In one example, the provided beverage compositions retain the same or about the same taste, odor and/or appearance as the same beverage composition that does not contain the composition according to embodiments disclosed herein. As such, dilution of a composition according to one or more embodiments disclosed herein results in a beverage or other consumable material which retains organoleptic properties desirable for consumption by an average human subject. Desirable and undesirable organoleptic properties can be measured by a variety of methods known to those skilled in the art, including, for example, organoleptic evaluation methods by which undesirable properties are detectable by sight, taste and/or smell and chemical tests, as well as by chemical analytical methods. As used herein, “retaining the organoleptic properties” refers to retention of these properties upon storage for a recited period of time, typically at room temperature.

As used herein, “visible particles” are particles, for example, in a liquid, such as an emulsion, that are visible when viewing the liquid with the naked eye (i.e., without magnification). For example, the visible particles can be particles that are observed by the artisan formulating embodiments of the cannabinoid enriched solid composition or the mixture produced by dilution thereof. In one example, the dilution of the cannabinoid enriched solid composition contain no visible particles. In another example, the diluted compositions contain few visible particles, for example, no more visible particles than another liquid, for example, a beverage. The presence of visible particles and the number of visible particles is determined by empirical observation.

As used herein, “clear” can be used to describe the resultant mixture upon dilution of the compositions provided herein.

As used herein, “stability” refers to a desirable property of the provided cannabinoid enriched solid composition and/or the liquid dilution of the cannabinoid enriched solid composition. For example, the ability of the provided cannabinoid enriched solid composition, or the liquid dilution of the cannabinoid enriched solid composition to remain free from one or more changes over a period of time, for example, at least or longer than 1 day, 1 week, 1 month, 1 year, or more. For example, a cannabinoid enriched solid composition can be described as stable if it is formulated such that it remains free from oxidation or substantial oxidation over time, and/or desirable for human consumption over time, has a lack of precipitates forming over time, does not exhibit any visible phase separation over a period of time.

As used herein, “stabilize” means to increase the stability of one of the provided compositions.

As used herein, “hydrophilic” and “polar” refer synonymously to ingredients and/or compounds having greater solubility in aqueous liquids, for example, water, than in fats, oils and/or organic solvents (e.g., methanol, ethanol, ethyl ether, acetone and benzene).

As used herein, a “solvent” is an ingredient that can be used to dissolve another ingredient. Solvents include polar and non-polar solvents. Non-polar solvents include oils and other non-polar ingredients that dissolve non-polar compounds. Typically, the non-polar solvent included in embodiments of the cannabinoid enriched solid composition or the mixture produced by dilution thereof is an oil. The non-polar solvent typically is not the non-polar ingredient itself, i.e., is distinct from the cannabinoid. More than one non-polar solvent can be used. Certain compounds, for example, flaxseed oil and safflower oil, can be non-polar solvents and non-polar ingredients. Typically, the non-polar solvent contains one or more oils, typically oils other than the non-polar ingredient or oil(s) not contained in the non-polar ingredient. Exemplary non-polar solvents include, but are not limited to, oils (in addition to the non-polar ingredient), for example, tocopheryl polyalkylene glycol oil, flaxseed oil, CLA, borage oil, rice bran oil, D-limonene, canola oil, corn oil, MCT oil and oat oil. Other oils also can be used.

As used herein, “polar solvent” refers to a solvent that is readily miscible with water and other polar solvents. Polar solvents are well-known and can be assessed by measuring any parameter known to those of skill in the art, including dielectric constant, polarity index and dipole moment (see, e.g., Przybitek (1980) “High Purity Solvent Guide,” Burdick and Jackson Laboratories, Inc.). For example, polar solvents generally have high dielectric constants, such as greater than or about 15, generally have high polarity indices, typically greater than or about 3, and generally large dipole moments, for example, greater than or about 1.4 Debye. Polar solvents include polar protic solvents and polar aprotic solvents.

As used herein, a “polar protic solvent” is a polar solvent containing a hydrogen atom attached to an electronegative atom, such that the hydrogen has a proton-like character and/or the bond between the hydrogen and electronegative atom is polarized. Exemplary polar protic solvents include, but are not limited to, water, alcohols, including monohydric, dihydric and trihydric alcohols, including, but not limited to, methanol, ethanol, glycerin and propylene glycol.

As used herein, “monohydric alcohols” are alcohols that contain a single hydroxyl group including, but not limited to, methanol, ethanol, propanol, isopropanol, n-butanol and t-butanol.

As used herein, “dihydric alcohols” are alcohols that contain two hydroxyl groups. Exemplary dihydric alcohols include, but are not limited to, glycols, e.g., propylene glycol, ethylene glycol, tetraethylene glycol, triethylene glycol and trimethylene glycol.

As used herein, “trihydric alcohols” are alcohols that contain three hydroxyl groups. Exemplary trihydric alcohols include, but are not limited to, glycerin, butane-1,2,3-triol, pentane-1,3,5-triol and 2-amino-2-hydroxymethyl-propane-1,3-diol.

As used herein, “non-polar,” “lipophilic” and “lipid-soluble” synonymously refer to compounds and/or ingredients, for example, non-polar compounds and non-polar ingredients, which have greater solubility in organic solvents (e.g., ethanol, methanol, ethyl ether, acetone and benzene), fats and oils than in aqueous liquids, for example, water. Non-polar ingredients include drugs, hormones, vitamins, nutrients and other lipophilic compounds. Typically, non-polar ingredients are poorly water-soluble, for example, water insoluble or compounds having low water solubility. Exemplary non-polar ingredients include ingredients that contain one or more non-polar compounds, for example, lipid-soluble drugs, hormones, essential fatty acids, for example, polyunsaturated fatty acids (PUFA), for example, omega-3 and omega-6 fatty acids, vitamins, nutrients, nutraceuticals, minerals and other compounds. Additional exemplary non-polar ingredients are described herein. The provided compositions can be formulated with any non-polar ingredient, for example, any non-polar ingredient that is or contains a non-polar compound.

As used herein, an “additive” includes anything other than cannabinoids that one can add to a food, beverage, or other human consumable to enhance one or more of its nutritional, pharmaceutical, dietary, health, nutraceutical, health benefit, energy-providing, treating, holistic, or other properties. For example, the additives can be oil-based additives (e.g., non-polar ingredients), such as nutraceuticals; pharmaceuticals; vitamins, for example, oil-soluble vitamins, e.g., vitamin D, tocopheryl polyalkylene glycol and vitamin A; minerals; fatty acids, such as essential fatty acids, for example, polyunsaturated fatty acids, e.g., omega-3 fatty acids and omega-6 fatty acids, such as alpha-linolenic acid (ALA), docosahexaenoic acid (DHA), eicosapentaenoic acid (EPA), gamma-linolenic acid (GLA), CLA, saw palmetto extract, flaxseed oil, fish oil and algae oil. phytosterols; coenzymes, such as coenzyme Q10; and any other oil-based additives.

As used herein, “water insoluble” refers to a compound that does not dissolve when the compound is mixed with water, for example, when mixed with water at room temperature, for example, between or between about 25° C. and 50° C.

As used herein, “low water solubility” refers to a compound that has a solubility in water of less than or about 30 mg/mL, for example, when mixed with water at room temperature, such as between or between about 25° C. and 50° C. As used herein, “poorly water-soluble” can be used to refer to compounds, for example, non-polar compounds, that are water insoluble or have low water solubility.

As used herein, “foodstuff” refers to a material which may be consumed by a mammal, preferably a human being, likewise “food and beverage product” refers to a product that is suitable for human consumption. For example, “food and beverage product” and/or “foodstuff” can refer to a composition that is dissolved in a solvent, typically an aqueous solvent, e.g., water, to form a liquid dilution composition, i.e., beverage composition or beverage product. “Food and beverage product” and/or “foodstuff” can also refer to a component of a final product that is suitable for human consumption, and/or the final product suitable for human consumption, such as the liquid dilution composition, i.e., beverage composition or beverage product.

As used herein, a “beverage base” refers to an aqueous composition to which one or more non-polar ingredients can be added. A beverage base includes, but is not limited to, an aqueous composition that contains one or more of a polar solvent, typically water, a juice, such as a fruit juice, a fruit juice concentrate, a fruit juice extract, a fruit flavor, a soda, a flavored soda, a carbonated water, a carbonated juice and any combination thereof. Embodiments of the cannabinoid enriched solid composition can be introduced into a beverage base (or beverage or other food).

As used herein, a “fruit juice,” “fruit juice concentrate,” “fruit juice extract” or “fruit flavor” refer to fruit-based juices and flavors that impart taste or smell to the provided beverage compositions (products). Any juice or fruit flavor can be added to the provided beverage compositions, including, but not limited to, plum, prune, date, currant, fig, grape, raisin, cranberry, pineapple, peach, nectarine, banana, apple, pear, guava, apricot, Saskatoon berry, blueberry, plains berry, prairie berry, mulberry, elderberry, Barbados cherry (acerola cherry), choke cherry, chocolate, vanilla, caramel, coconut, olive, raspberry, strawberry, huckleberry, loganberry, dewberry, boysenberry, kiwi, cherry, blackberry, honey dew, green tea, cucumber, quince, buckthorn, passion fruit, sloe, rowan, gooseberry, pomegranate, persimmon, mango, rhubarb, papaya, litchi, lemon, orange, lime, tangerine, mandarin and grapefruit juices, or any combination thereof. Exemplary beverage compositions provided herein include combinations of juices or flavors that impart peach mango, peach, citrus, pomegranate blueberry, tropical berry, cherry chocolate, vanilla, cherry vanilla, chocolate blueberry, chocolate caramel, cucumber, green tea, honey-dew melon, pineapple papaya, peach nectarine, raspberry lemonade, grape, orange tangerine, orange, lime and mixed berry flavors.

As used herein, “fatty acid” refers to straight-chain hydrocarbon molecules with a carboxyl (—COOH) group at one end of the chain.

As used herein, “polyunsaturated fatty acid” and “PUFA” are used synonymously to refer to fatty acids that contain more than one carbon-carbon double bonds in the carbon chain of the fatty acid. PUFAs, particularly essential fatty acids, are useful as dietary supplements.

Examples include omega-3 fatty acids such as alpha-linolenic acid (alpha-linolenic acid; ALA) (18:3omega3) (a short-chain fatty acid); stearidonic acid (18:4omega3) (a short-chain fatty acid); eicosapentaenoic acid (EPA) (20:5omega3); docosahexaenoic acid (DHA) (22:6omega3); eicosatetraenoic acid (24:4omega3); docosapentaenoic acid (DPA, clupanodonic acid) (22:5omega3); 16:3 omega3; 24:5 omega3 and nisinic acid (24:6omega3). Longer chain omega-3 fatty acids can be synthesized from ALA (the short-chain omega-3 fatty acid). Exemplary of non-polar ingredients containing omega-3 fatty acids are non-polar ingredients containing DHA and/or EPA, for example, containing fish oil, krill oil and/or algae oil, for example, microalgae oil, and non-polar ingredients containing alpha-linolenic acid (ALA), for example, containing flaxseed oil. Other exemplary fatty acids include linoleic acid (18:2omega6) (a short-chain fatty acid); gamma-linolenic acid (GLA) (18:3omega6); dihomo gamma linolenic acid (DGLA) (20:3omega6); eicosadienoic acid (20:2omega6); arachidonic acid (AA) (20:4omega6); docosadienoic acid (22:2omega6); adrenic acid (22:4omega6); and docosapentaenoic acid (22:5omega6). Exemplary of non-polar ingredients containing omega-6 fatty acids are ingredients containing GLA, for example, borage oil. Also exemplary of omega-6-containing non-polar ingredients are compounds containing conjugated fatty acids, for example, conjugated linoleic acid (CLA) and compounds containing saw palmetto extract.

As used herein, “preservative” and “preservatives” are used synonymously to refer to ingredients that can improve the stability of embodiments of the cannabinoid enriched solid composition and/or the liquid produced by dilution of the cannabinoid enriched solid composition. Preservatives, particularly food and beverage preservatives, are well known. Any known preservative can be used in embodiments of the cannabinoid enriched solid composition and/or the liquid produced by dilution of the cannabinoid enriched solid composition. Exemplary of the preservatives include benzyl alcohol, benzyl benzoate, methyl paraben, propyl paraben, antioxidants, for example, vitamin E, vitamin A palmitate and beta carotene. Typically, a preservative is selected that is safe for human consumption, for example, in foods and beverages, for example, a GRAS certified and/or Kosher-certified preservative, for example, benzyl alcohol.

As used herein, an “antioxidant” refers to a stabilizer or one component of a stabilizing system that acts as an antioxidant, and that, when embodiments of the cannabinoid enriched solid composition are added to a beverage composition in combination with the other required components (i.e., acid and/or bicarbonate or carbonate) yields beverage compositions that retain one or more desired organoleptic properties, such as, but not limited to, the taste, smell, odor and/or appearance, of the beverage composition over time. Typically, antioxidants are food-approved, e.g., edible antioxidants, for example, antioxidants that are safe and/or approved for human consumption. Exemplary antioxidants include, but are not limited to, ascorbic acid, vitamin C, ascorbate and coenzyme Q-containing compounds, including, but not limited to, coenzyme Q10.

As used herein, an “acid” or “ingestible acid” refers to a stabilizer or one component of a stabilizing system that, when added to a beverage composition in combination with the other components (i.e., antioxidant and/or bicarbonate or carbonate), yields compositions that retain one or more desired organoleptic properties, such as, but not limited to, the taste, smell, odor and/or appearance of the composition over time. Typically, the acids are food-approved, e.g., edible acids or ingestible acids, for example, acids that are safe and/or approved for human consumption. Exemplary acids include, but are not limited to, citric acid, phosphoric acid, adipic acid, ascorbic acid, lactic acid, malic acid, fumaric acid, gluconic acid, succinic acid, tartaric acid and maleic acid.

As used herein, a “bicarbonate” or “carbonate” refers to a stabilizer or one component of a stabilizing system that, when added to a beverage composition in combination with the other components (i.e., the acid and/or antioxidant) yields compositions that retain one or more desired organoleptic properties, such as, but not limited to, the taste, smell, odor and/or appearance of the composition over time. Typically, bicarbonates or carbonates are food-approved, e.g., edible bicarbonates or carbonates, for example, bicarbonates or carbonates that are safe and/or approved for human consumption. Exemplary bicarbonates include, but are not limited to, potassium bicarbonate and sodium bicarbonate. Exemplary carbonates include, but are not limited to, potassium carbonate, sodium carbonate, calcium carbonate, magnesium carbonate and zinc carbonate.

As used herein, “emulsion stabilizer” refers to compounds that can be used to stabilize and/or emulsify and/or change the viscosity of embodiments of the cannabinoid enriched solid composition and/or the liquid produced by dilution of the cannabinoid enriched solid composition. For example, the emulsion stabilizer can increase the viscosity of the liquid produced by dilution of the cannabinoid enriched solid composition. One or more emulsion stabilizers can be used. Addition of an emulsion stabilizer can prevent separation of the cannabinoid enriched solid composition and/or the liquid produced by dilution of the cannabinoid enriched solid composition.

As used herein, a “pH adjuster” is any compound, typically an acid or a base, that is capable of changing the pH of embodiments of the cannabinoid enriched solid composition or the mixture produced by dilution thereof, for example, to reduce the pH of the cannabinoid enriched solid composition and/or the liquid produced by dilution of the cannabinoid enriched solid composition, or to increase the pH of the same, typically without altering other properties of the cannabinoid enriched solid composition and/or the liquid produced by dilution of the cannabinoid enriched solid composition, or without substantially altering other properties. pH adjusters are well known. Exemplary of the pH adjusters are acids, for example, citric acid and phosphoric acid, and bases.

As used herein, “flavor” is any ingredient that changes, typically improves, the taste and/or smell of embodiments of the cannabinoid enriched solid composition and/or the liquid produced by dilution of the cannabinoid enriched solid composition, for example, in a beverage.

As used herein, “natural” is used to refer to a composition, and/or ingredients in embodiments of the cannabinoid enriched solid composition and/or the liquid produced by dilution of the cannabinoid enriched solid composition, that can be found in nature and is not solely man-made. For example, benzyl alcohol is a natural preservative. Similarly, tocopheryl polyethylene glycol is a natural surfactant. The natural composition/ingredient can be GRAS and/or Kosher-certified.

As used herein, “G.R.A.S.” and “GRAS” are used synonymously to refer to compounds, compositions and ingredients that are “Generally Regarded as Safe” by the USDA and FDA for use as additives, for example, in foods, beverages and/or other substance for human consumption, such as any substance that meets the criteria of sections 201(s) and 409 of the U.S. Federal Food, Drug and Cosmetic Act. Typically, embodiments of the cannabinoid enriched solid composition and/or the liquid produced by dilution of the cannabinoid enriched solid composition disclosed herein are GRAS certified. Likewise, “kosher” is used to refer to substances that conform to Jewish Kosher dietary laws, for example, substances that do not contain ingredients derived from non-kosher animals or do not contain ingredients that were not made following kosher procedures. Typically, embodiments of the cannabinoid enriched solid composition and/or the liquid produced by dilution of the cannabinoid enriched solid composition are Kosher-certified.

As used herein, “excipients”, refer to any substance needed to formulate the cannabinoid enriched solid composition to a desired form. For example, suitable excipients include but are not limited to, diluents or fillers, binders or granulating agents or adhesives, disintegrates, lubricants, antiadherants, glidants, wetting agents, dissolution retardants or enhancers, adsorbents, buffers, chelating agents, preservatives, colors, flavors and sweeteners. Typical excipients include, but are not limited to, starch, pregelatinized starch, maltodextrin, monohydrous dextrose, alginic acid, sorbitol and mannitol. In general, the excipient should be selected from non-toxic excipients (IIG, Inactive Ingredient Guide, or GRAS, Generally Regarded as safe, Handbook of Pharmaceutical Excipients).

As used herein, a binder is an excipient added to a composition to aid formation of a powder when the cannabinoid enriched solid composition is dried. Non-limiting examples of suitable binders include, but are not limited to, acacia, dextrin, starch, povidone, carboxymethylcellulose, guar gum, glucose, hydroxypropyl methylcellulose, methylcellulose, polymethacrylates, maltodextrin, hydroxyethyl cellulose, whey, disaccharides, sucrose, lactose, polysaccharides and their derivatives such as starches, cellulose or modified cellulose such as microcrystalline cellulose and cellulose ethers such as hydroxypropyl cellulose, sugar alcohols such as xylitol, sorbitol or maltitol, protein, gelatins and synthetic polymers, such as polyvinylpyrrolidone (PVP) or polyethylene glycol (PEG).

As used herein, “homolog” refers to an analog that differs from the parent compound only by the presence or absence of a simple unit, such as a methylene unit, or some multiple of such units, e.g., —(CH₂)ₙ—. Typically, a homolog has similar chemical and physical properties as the parent compound. Exemplary of the homologs used in the provided compositions and methods are TPGS homologs.

As used herein, pharmaceutical compositions comprising embodiments of the composition refer to compositions formulated for administration in a pharmaceutical carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition according to one or more aspects of this disclosure as a unit-dose formulation, for example, a tablet, which may contain from about 0.01 or 0.5% to about 95% or 99% by weight of the cannabinoid(s) component. Furthermore, a “pharmaceutically acceptable” component such as a salt, carrier, excipient or diluent of a composition according to the instant disclosure is a component that (i) is compatible with the other ingredients of the composition in that it can be combined with the compositions of the present disclosure without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

As used herein, a biocompatible, biodegradable polymer is a polymer which can be broken down in vivo to monomer and/or oligomer fragments, wherein the monomeric or oligomeric fragments do not provoke an immune response, are not toxic, and can be easily excreted.

In one or more embodiments, a method of treating a medical condition addressed by one or more cannabinoids, comprises the steps of providing a feed mixture comprising one or more cannabinoids to an animal capable of producing a foodstuff; collecting a cannabinoid enriched foodstuff produced by the animal; combining a portion of the cannabinoid enriched foodstuff with an amount of an absorbent, an adsorbent, or a combination thereof, to produce a free-flowing cannabinoid enriched solid composition, and providing the cannabinoid enriched solid composition to a recipient in an amount sufficient to treat the medical condition. In an embodiment, the cannabinoid enriched solid composition comprises greater than or equal to about 50 wt %, preferably greater than or equal to about 50 wt %, preferably greater than or equal to about 10 wt %, preferably greater than or equal to about 20 wt %, preferably greater than or equal to about 30 wt %, preferably greater than or equal to about 40 wt %, preferably greater than or equal to about 50 wt %, preferably greater than or equal to about 60 wt %, preferably greater than or equal to about 70 wt %, preferably greater than or equal to about 90 wt % of the cannabinoid enriched foodstuff and the balance of the composition being the absorbent and/or adsorbent carrier. In a preferred embodiment, the cannabinoid enriched solid composition comprises from about 20 wt % to about 60 wt % of the cannabinoid enriched foodstuff.

In embodiments, the animal is a bee and the foodstuff is honey. Suitable examples of bees include Bombus terrestris, Apis mellifera, and the like. Suitable methods for producing the cannabinoid enriched foodstuff include those disclosed in WO2017/013661, the contents of which are fully incorporated by reference herein.

In other embodiments, the foodstuff is produced by feeding an animal, e g, an insect, a composition comprising cannabinoids, e.g., hemp, followed by harvesting the animals and processing at least a portion of the animal corpses to produce the foodstuff.

In embodiments, the absorbent and/or the adsorbent comprise a maltodextrin, a nano porous starch aerogel, an agglomerated starch comprising a starch binding agent and starch granules, a thermally inhibited agglomerated starch, an amylopectin, a modified waxy maize starch, or a combination thereof. Suitable examples of absorbents include those disclosed in US20190136017, US20190000101, US20180343883, US20180327767, US20180209101, US20180209100, US20180192683, US20170283818, US20170282524, US20170282523, US20170282522, US20170282519, US20170282487, US20160053027, US20150201654 US20150083024, and US20150045454, the disclosures of which are fully incorporated by reference herein.

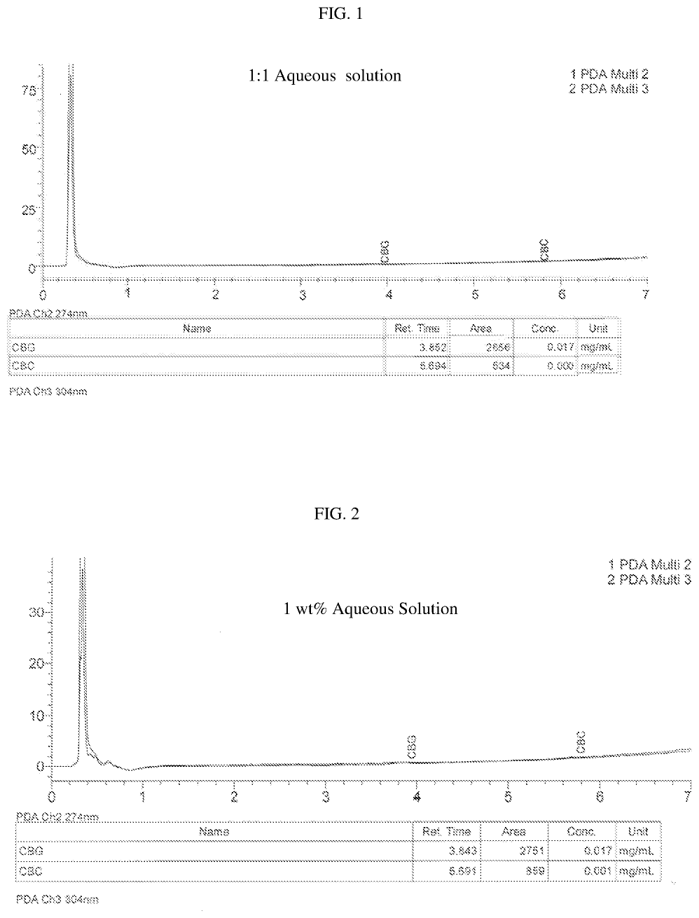

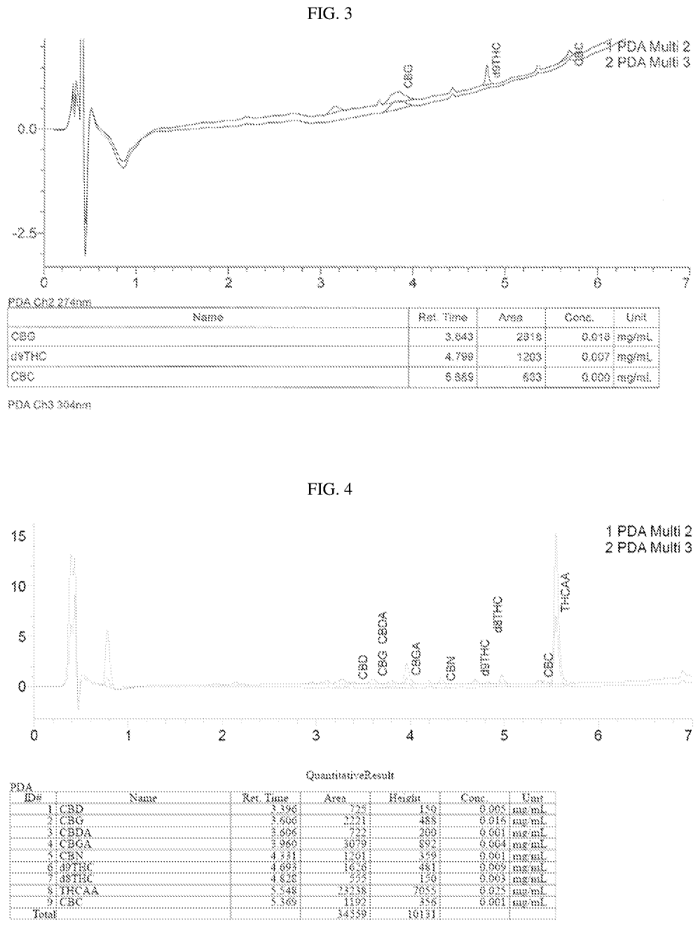

In one or more embodiments, the feed mixture fed to the animal comprises one or more cannabinoids, preferably tetrahydrocannabinol (THC), cannabidiol (CBD), or a combination thereof. In embodiments, the feed mixture comprises from about 0.1 wt % to about 5 wt % of the one or more cannabinoids, based on the total amount of the feed mixture present. However, in embodiments, the concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the composition present. This is shown in FIGS. 1, 2, and 3, wherein compositions comprising the cannabinoid enriched honey show miniscule concentrations of free cannabinoids while still possessing bioactive properties.

Accordingly, it is believed that the cannabinoids are present as various water soluble or water dispersible conjugates in the cannabinoid enriched foodstuff and subsequently in the cannabinoid enriched solid composition. In embodiments, at least some of the cannabinoids present in the cannabinoid enriched solid composition are present as a water soluble conjugate, a water dispersible conjugate, or a combination thereof. In embodiments, the cannabinoids are present in the cannabinoid enriched solid composition as glycosides, as glutathione conjugates, or a combination thereof.

In one or more embodiments, a cannabinoid enriched composition comprises a cannabinoid enriched foodstuff produced by the living organism in the animal kingdom; in combination with an absorbent, an adsorbent, or a combination thereof, present in an amount such that the composition is a free-flowing solid. In embodiments, the cannabinoid enriched foodstuff is honey produced by bees which have been fed a feed mixture comprising one or more cannabinoids.

In one or more embodiments, the absorbent, the adsorbent, or both, comprise a maltodextrin, a nanoporous starch aerogel, an agglomerated starch comprising a starch binding agent and starch granules, a thermally inhibited agglomerated starch, an amylopectin, a modified waxy maize starch, or a combination thereof.

In embodiments, a concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the cannabinoid enriched composition present. For purposes herein, free THC, CBD, or both refers to the detection of the cannabinoids via HPLC-UV detection.

In embodiments, the cannabinoids are present in the cannabinoid enriched composition as water soluble and/or water dispersible conjugates, derivatives, metabolites, or a combination thereof.

In embodiments, the cannabinoids are present in the cannabinoid enriched solid composition bonded to one or more carbohydrate moieties e.g., glycosides, as conjugates, e.g., a glutathione conjugates, or a combination thereof.

In one or more embodiments, the absorbent and/or the adsorbent may comprise fibrinogen; dextran; cellulose including cellulose, cellulose acetate, cellulose butyrate, cellulose acetate butyrate, cellophane, cellulose nitrate, cellulose propionate, cellulose ethers, and carboxymethyl cellulose; starch; pectin; chitosan; gelatin; alginate and conjugations thereof including alginate-gelatin, alginate-collagen, alginate-laminin, alginate-elastin, alginate-collagen-laminin and alginate-hyaluronic acid; collagen and conjugates thereof; hyaluronan; hyaluronic acid; sodium hyaluronate; modified hyaluronan such as tyramine-hyaluronate or glycidyl methacrylate hyaluronate; or self-assembled peptides including dipeptides, so called “lego peptides, ionic self-complementary peptides, surfactant peptides, molecular paint peptides, carpet peptides, cyclic peptides, and the like.

In one or more of the embodiments, the combination of the absorbent and/or the adsorbent and the cannabinoid enriched solid composition are processed at reduced temperatures via chilling and/or freezing utilizing refrigeration and/or freezing apparatus or via the use of cryogenic liquids, e.g., liquid nitrogen or utilizing solid carbon dioxide, to achieve sufficient embrittlement to allow for grinding, pulverizing or other mechanical process so that adequate particle size reduction and a uniform particle size is achieved. Chilling, freezing or the use of cryogenic liquids or other solids not only achieves embrittlement to facilitate the fracturing of the absorbent and/or the adsorbent and the cannabinoid enriched solid composition, but also prevents the build-up of heat preventing the separation of the cannabinoid enriched solid composition out of the absorbent and/or the adsorbent, thus preventing agglomeration e.g., clumping, of the material upon mechanical grinding or other similar pulverization or mastication processes to reduce the particle size and to create a uniform combination of the absorbent and/or the adsorbent and the cannabinoid enriched solid composition.

Suitable cannabinoids for use herein include both optically pure and racemic pairs of compounds which may be isolated from one or more of the Cannabis sativa plants including chemotypes I, II, III, and the like. Suitable cannabinoids for purposes herein may be isolated from the Cannabis sativa plant and/or may be synthetically produced and/or modified, and/or biosynthesized. Unless explicitly stated otherwise, the term “cannabinoids” refers to one or more of the cyclized and/or uncyclized, substituted and/or unsubstituted forms of: cannabigerol, according to the general formula:

i) cannabigerol, according to the general formula:

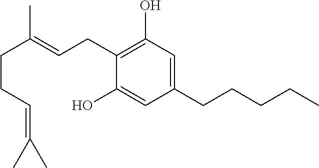

ii) cannabichromene, according to the general formula:

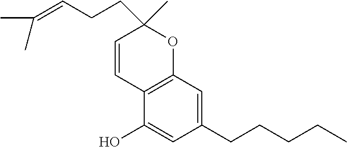

iii) cannabidiol, according to the general formula:

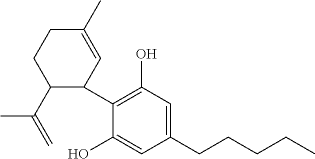

iv) tetrahydrocannabinol and/or cannabinol, according to the general formula:

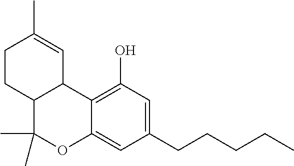

v) cannabielsoin, according to the general formula:

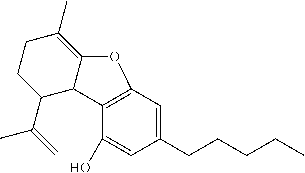

vi) iso-tetrahydrocannabinol, according to the general formula:

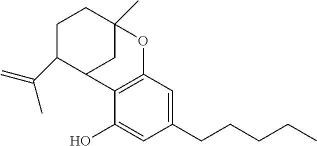

vii) cannabicyclol, according to the general formula:

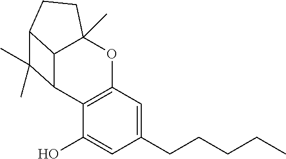

viii) cannabicitran, according to the general formula:

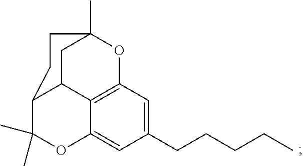

ix) tetrahydrocannabivarin (THCV), according to the general formula:

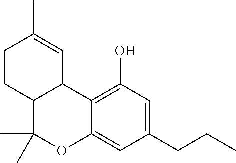

wherein any one or more of the various hydrogen atoms may be substituted with a functional group, and/or including the free acids, salts, tosylates, mesylates, esters, amides, ethers, sulfates, and/or other derivatives thereof.

In one aspect of the disclosure, the final solid product includes one or more tetrahydrocannabinols (THC) in general, and (−)-trans-Δ⁹-tetrahydrocannabinol in particular, cannabidol (CBD), tetrahydrocannabinolic acid (THCA), cannabidiolic acid (CBDA), cannabinol (CBN), cannabigerol (CBG), cannabichromene (CBC) cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), cannabigerolic acid, cannabigerolic acid monomethylether, cannabigerol monomethylether, cannabigerovarinic acid, cannabichromenic acid, cannabichromevarinic acid, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-isotetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxycannabitriolvarin, dehydrocannabifuran, cannabifuran, cannabichromanon, cannabicitran, 10-oxo-delta-6a-tetrahydrocannabinol, delta-9-cistetrahydrocannabinol, 3,4,5,6-tetrahydro-7-hydroxy-alpha-alpha-2-trimethyl-9-npropyl-2,6-methano-2H-1-benzoxocin-5-methanol-cannabiripsol, trihydroxy-delta-9-tetrahydrocannabinol, cannabinol, and/or derivatives thereof.

In one or more aspects the phenolic hydrogen, when present, is replaced by a C₁-C₄₀ hydrocarbyl, preferably a C₃-C₄₀ carbohydrate, saccharide or polysaccharide, optionally comprising one or more functional groups, e.g., an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more aspects the cannabinoid comprises both substituted and unsubstituted forms of cannabidiol (CBD) according to the general formula:

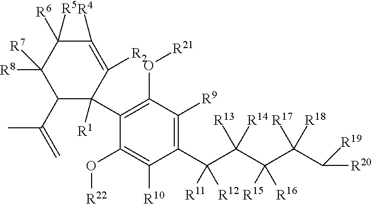

wherein one or more of R¹, R², R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², are independently selected from hydrogen or one or more monovalent radicals including hydrocarbyl radicals such as methyl, ethyl, ethenyl, and all isomers (including cyclics such as cyclohexyl) of propyl, butyl, pentyl, hexyl, heptyl, octyl, nonyl, decyl, undecyl, dodecyl, propenyl, butenyl, and from halocarbyls and all isomers of halocarbyls including perfluoropropyl, perfluorobutyl, perfluoroethyl, perfluoromethyl, and from substituted hydrocarbyl radicals and all isomers of substituted hydrocarbyl radicals including trimethylsilylpropyl, trimethylsilylmethyl, trimethylsilylethyl, and from phenyl, and all isomers of hydrocarbyl substituted phenyl including methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, dipropylmethylphenyl, and the like; from all isomers of halo substituted phenyl (where halo is, independently, fluoro, chloro, bromo and iodo) including halophenyl, dihalophenyl, trihalophenyl, tetrahalophenyl, and pentahalophenyl; and from all isomers of halo substituted hydrocarbyl substituted phenyl (where halo is, independently, fluoro, chloro, bromo and iodo) including halomethylphenyl, dihalomethylphenyl, (trifluoromethyl)phenyl, bis(triflouromethyl)phenyl; and from all isomers of benzyl, and all isomers of hydrocarbyl substituted benzyl including methylbenzyl, dimethylbenzyl.

In one or more aspects R²¹ and/or R²², comprise a C₃-C₄₀ carbohydrate, saccharide or polysaccharide, optionally comprising one or more functional groups, e,g, an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more aspects, one or more of R¹, R², R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², is substituted with one or more functional groups selected from Br, Cl, F, I, —NR*₂, —NR*—CO—R*, —OR*, *—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

In embodiments, at least a portion of the cannabinoid is present in the composition as a glucose and/or other carbohydrate conjugate, i.e., a glycoside, and/or as a glutathione conjugate, such as a glutathione S-conjugate. As used herein, a glutathione conjugate of a cannabinoid is a cannabinoid comprising a moiety derived from (2S)-2-Amino-4-{[(1R)-1-[(carboxymethyl)carbamoyl]-2-sulfanylethyl]carbamoyl}butanoic acid.

The cannabinoids may be isolated from plants, e.g., Cannabis sativa, and/or may be produced synthetically, and/or may be isolated from plants and subsequently modified via natural and/or synthetic means, and/or derivatized according to one or more embodiments disclosed herein.

In one or more embodiments, the phenolic hydrogen, when present, is replaced by a C₁-C₄₀ hydrocarbyl, preferably a C₃-C₄₀ carbohydrate, saccharide or polysaccharide, optionally comprising one or more functional groups, e.g., an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more embodiments, R²⁰ and/or R²¹ is substituted with monovalent functional group comprising a triose, a tetrose, a pentose, a hexose, a heptose, a glycoside, and/or a combination thereof.

In embodiments, the compositions according to the instant disclosure may further comprise one or more surfactants and/or a surfactant system. In one or more embodiments the composition further comprises one or more surfactants and/or a surfactant system. In some embodiments, the surfactant is a phospholipid, a sugar fatty acid ester, a sucrose fatty acid ester, a polysorbate and a polysorbate analog, or a combination thereof. In one or more embodiments of the composition, at least one surfactant has an HLB of greater than or equal to about 10 and/or further comprises at least one surfactant having an HLB of less than 10. In one or more embodiments the surfactant system comprises a matched pair suitable for aqueous dispersion.

In one or more embodiments the composition further comprises an emulsion stabilizer selected from the group consisting of xanthan gum, guar gum and sodium alginate; modified gum acacia; ester gum, or a combination thereof, and/or a pH adjuster present in an amount sufficient to adjust the pH of 1 wt % of the composition in deionized water to greater than or equal to about 6 and less than or equal to about 8 at 25° C.

The solid composition may further comprise a hydrophilic component, e.g., comprising one or more water soluble agents. Exemplary water soluble agents include, but are not limited to, carbohydrates, including complex carbohydrates such as starches, gum arabic, and quillaja extract; sugars such as monosaccharides (e.g., dextrose), oligosaccharides (e.g., cyclodextrins), and polysaccharides (e.g., maltodextrin); and polyols including, e.g., sugar alcohols such as sorbitol and maltitol. Additional water soluble agents that may be used herein include proteins (e.g., gelatin, whey, casein), phospholipids (e.g., soy lecithin, egg lecithin, etc.), glycerol monostearate, surfactants (such as, e.g., sorbitan, sorbitan esters, and polysorbates (e.g., sorbitan monolaurate, polyoxyethylene (20) sorbitan monolaurate, sorbitan monopalmitate, polyoxyethylene (20) sorbitan monopalmitate, sorbitan monostearate, polyoxyethylene (20) sorbitan monostearate, sorbitan tristearate, sorbitan monooleate, polyoxyethylene (20) sorbitan monooleate, etc.), and other emulsifiers and water soluble agents suitable for human consumption. The water soluble agent(s) may have a chemical structure that includes a hydrophilic region to promote solubility. Without intending to be bound by theory, it is believed that the water soluble agent(s) may promote solubility of the cannabinoid compounds, e.g., by at least partially absorbing the cannabinoid compounds or otherwise associating the cannabinoid compounds with hydrophilic portions of the water soluble agent.

Examples of water soluble agents include those comprising one or more complex carbohydrates, including e.g., natural carbohydrates such as starches, gum arabic, and quillaja extract. The starch may be a food starch (e.g., waxy maize, corn, potato, wheat, tapioca, or cassava, etc.), and may be relatively high in amylopectin and/or chemically modified to increase an oil absorption capacity of the starch. Examples of starches suitable for the compositions herein include different types of modified food starches, including, but not limited to, octenyl succinic anhydride (OSA) starch. In some examples, the composition may comprise at least one complex carbohydrate in combination with one or more other water soluble agents, such as, e.g., oligosaccharides, polysaccharides, surfactants, and/or polyols. Further, for example, the composition may comprise two or more different complex carbohydrates, optionally in combination with one or more oligosaccharides, polysaccharides, surfactants, and/or polyols. Commercial examples of water soluble agents suitable for the compositions and methods herein include, but are not limited to, CAPSUL®. starch, PURITY GUM® starch, N-ZORBIT® starch, PENBIND® starch, N-Lite® LP starch, and Q-Naturale® quillaja extract produced by Ingredion; and Span® 20, Span® 40, Span® 60, Span® 80, Tween® 20, Tween® 40, Tween® 60, and Tween® 80, produced by Croda International PLC.

In some embodiments, the composition comprises at least one water soluble agent chosen from a complex carbohydrate, a polyol, a polysaccharide, an oligosaccharide, or a combination thereof. For example, the water soluble agent(s) may comprise a starch, quillaja extract, maltodextrin, a sugar alcohol, or a combination thereof. In at least one example, the water soluble agent(s) comprise a modified food starch, sorbitol, or both. According to some aspects of the present disclosure, the composition comprises at least two water soluble agents. For example, the composition may comprise two or more different water soluble agents chosen from complex carbohydrates, polyols, polysaccharides, oligosaccharides, and combinations thereof. Further, for example, the two or more different water soluble agents may be chosen from modified food starches, sugar alcohols, quillaja extract, maltodextrin, or combinations thereof. In some examples, the two different water soluble agents comprise a starch and a sugar alcohol.

Certain water soluble agent(s) may provide sweetness to the composition. For example, sorbitol is a sugar alcohol that is generally understood to be metabolized at a slower rate than sugar, and thus may be described as a sugar substitute. Further, for example, maltodextrin is a long-chain polysaccharide that may be described as moderately sweet. In general, a longer chain length corresponds to a composition with less sweetness. For example, the water soluble agent(s) may comprise a polysaccharide or oligosaccharide that does not provide any sweetness, e.g., a polysaccharide or oligosaccharide that is flavorless.

In some examples herein, the weight ratio of water soluble agent(s) to hydrophobic component (i.e., purified oil distillate and carrier oil(s), if any) may range from about 10:1 to about 1:100, such as from about 5:1 to about 1:50, from about 4:1 to about 1:20, from about 3:1 to about 1:15, from about 2:1 to about 1:10, or from about 4:1 to about 1:4, e.g., a ratio of about 10:1, 5:1, 4:1, 3:1, 5:2, 2:1, 4:3, 1:1, 3:4, 1:2, 2:5, 1:3, 1:4, 1:5, 1:10, 1:25, 1:50, 1:75, or 1:100. In some examples, the weight ratio of water soluble agent(s) to the hydrophobic component ranges from about 1:5 to about 2:1, e.g., a weight ratio of up to about 1:1, up to about 1:2, up to about 1:3, up to about 1:4, or up to about 1:5.

In embodiments, the surfactant is a phospholipid, a sugar fatty acid ester, a sucrose fatty acid ester, a polysorbate and a polysorbate analog, or a combination thereof. In some embodiments, at least one surfactant has an HLB of greater than or equal to about 10 and/or may further comprise at least one surfactant having an HLB of less than 10.

The compositions may further include one or more other or co-surfactants to improve emulsification of the cannabinoid and/or the stability of the composition, for example, by preventing or slowing oxidation of the cannabinoid or other ingredient.

Suitable surfactants include phospholipids, for example, phosphatidylcholine. Other exemplary surfactants include non-ionic surfactants, such as sugar-derived surfactants, including fatty acid esters of sugars and sugar derivatives, and PEG-derived surfactants, such as PEG derivatives of sterols, PEG derivatives of fat-soluble vitamins and PEG-sorbitan fatty acid esters. Polyethylene/polypropylene/polybutene glycols may also be used.

When present, the amount of the surfactant is typically less than or equal to about 10 wt %, typically less than or less than about 5%, for example, the total amount of surfactant as a percentage (%), by weight, of the composition can be, e.g., less than or less than about 10%, such as less than or about 5%, 4.5%, 4%, 3.5%, 3.15%, 3%, 2.5%, 2%, 1.75%, 1.5%, 1.25%, 1%, 0.75%, 0.5%, 0.25%, 0.15% or less, by weight, of the total composition.

Suitable phospholipids include, but are not limited to lecithin, including phosphatidylcholine (PC), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM) or a combination thereof. Typically, the phospholipid is phosphatidylcholine (PC), which sometimes is referred to by the general name “lecithin.” Exemplary of the phospholipids that can be used as co-surfactants in the provided compositions are the phospholipids sold by Lipoid, LLC (Newark, N.J.), for example, Purified Egg Lecithins, Purified Soybean Lecithins, Hydrogenated Egg and Soybean Lecithins, Egg Phospholipids, Soybean Phospholipids, Hydrogenated Egg and Soybean Phospholipids, Synthetic Phospholipids, PEG-ylated Phospholipids and phospholipid blends. Exemplary of the phosphatidylcholine that can be used as a co-surfactant in the provided compositions is the phosphatidylcholine composition sold by Lipoid, LLC, under the name Lipoid S100, which is derived from soy extract and contains greater than or greater than about 95% phosphatidylcholine.

Suitable sugar-derived surfactants include, but are not limited to, sugar fatty acid esters including fatty acid esters of sucrose, glucose, maltose and other sugars, esterified to fatty acids of varying lengths (e.g., containing a varying numbers of carbons). The fatty acids typically have carbon chains between 8 and 28 carbons in length, and typically between 8 and 20, or between 8 and 18 or between 12 and 18, such as, but not limited to, stearic acid (18 carbons), oleic acid (18 carbons), palmitic acid (16 carbons), myristic acid (14 carbons) and lauric acid (12 carbons). Typically, the sugar ester surfactants are sucrose ester surfactants, typically sucrose fatty acid ester surfactants.

Suitable polyalkylene derived surfactants include, but are not limited to PEG derivatives of sterols, e.g., a cholesterol or a sitosterol (including, for example, any of the PEG derivatives disclosed in U.S. Pat. No. 6,632,443); PEG derivatives of fat-soluble vitamins, for example, some forms of vitamin A (e.g., retinol) or vitamin D (e.g., vitamin D1-D5); and PEG-sorbitan fatty acid esters, such as polysorbates, including polyoxyethylene (20) sorbitan monooleate (also called polysorbate 80) and analogs (e.g., homologs) of polysorbate 80, such as, for example, polysorbate 20 (polyoxyethylene (20) sorbitan monolaurate), polysorbate 40 (polyoxyethylene (20) sorbitan monopalmitate) and polysorbate 60 (polyoxyethylene (20) sorbitan monostearate); and stearic acid derivatives, including, for example, polyethylene glycol 400 distearate (PEG 400 DS), such as the PEG 400 DS sold by Stepan Lipid Nutrition (Maywood, N.J.).

Suitable sugar fatty acid ester surfactants include sucrose fatty acid esters wherein the fatty acid contains between 4 and 28 carbon atoms, typically between 8 and 28 carbon atoms, and typically between 8 and 25 carbon atoms, such as between 8 and 18 carbon atoms, such as 8, 9, 10, 11, 12, 13, 14, 15, 16, 17 and 18 carbon atoms. The fatty acid can be synthetic or naturally occurring and include linear and branched fatty acids. The fatty acids include, but are not limited to, myristic acid, palmitic acid, stearic acid, oleic acid, caproic acid, capric (or decanoic) acid, lauric acid, caprylic acid and pelargonic (or nonanoic) acid.

In embodiments, the sugar fatty acid ester is a sucrose fatty acid ester surfactant which may be sucrose monoesters, diesters, triesters and polyesters, and mixtures thereof, and typically contain sucrose monoesters. The sucrose fatty acid ester surfactants include single fatty acid esters and also include homogeneous mixtures of sucrose esters, containing members with different lengths of fatty acid carbon chain and/or members with different degrees of esterification. For example, the sucrose fatty acid ester surfactants include mixtures of monoesters, diesters, triesters, and/or polyesters. The sugar ester surfactants further include sucrose fatty acid ester analogs and homologs and mixtures thereof.

Suitable sucrose fatty acid esters include mixtures of sucrose fatty acid esters, and may have varying HLB values, such as HLB values ranging from at or about 1 to at or about 20. The HLB value of the sucrose fatty acid ester generally depends on the degree of esterification (e.g., the average degree of esterification in a mixture of different esters). Typically, the lower the degree of esterification (e.g., average degree), the higher the HLB value of the sucrose fatty acid ester or mixture thereof. Exemplary sucrose esters include sucrose distearate (HLB=3), sucrose distearate/monostearate (HLB 12), sucrose dipalmitate (HLB=7.4), sucrose monostearate (HLB=15), sucrose monopalmitate (HLB>10), sucrose monolaurate (HLB 15). Typically, the sucrose fatty acid ester surfactants in embodiments of the cannabinoid enriched solid composition have an HLB value of between at or about 13 and at or about 20, such as at or about 13, 14, 15, 16, 17, 18, 19, or 20, and typically between at or about 13 and at or about 18, such as, but not limited to, HLB values of at or about 15, 16 and 17, such as, for example, sucrose ester surfactants including sucrose monopalmitate, sucrose monolaurate and sucrose monostearate.

In embodiments the sucrose ester mixtures have at least at or about 50%, by weight (w/w), monoester, such as at least or about at least 55, 60, 65, 70, 75, 80, 85, 90, 95, 97, 98, 99 or 100%, by weight (w/w), sucrose monoesters, and typically at least at or about 60%, by weight, or at least at or about 70%, by weight (w/w), monoesters.

Suitable examples of sucrose fatty acid ester surfactants include sucrose fatty acid monoesters, such as sucrose monocaprylate, sucrose monodecanoate, sucrose monolaurate, sucrose monomyristate, sucrose monopalmitate, sucrose monostearate, sucrose monopelargonate, sucrose monoundecanoate, sucrose monotridecanoate, sucrose monopentadecanoate and sucrose monoheptadecanoate. The sucrose fatty acid esters further include mixtures containing varying percentages of monoesters, diesters, triesters and polyesters, such as, but not limited to, a mixture having at or about 72% monoesters, 23% diesters, 5% triesters and 0 polyesters; a mixture having at or about 61% monoesters, 30% diesters, 7% triesters, and 2% polyesters; and a mixture having at or about 52% monoesters, 36% diesters, 10% triesters and 2% polyesters.

In embodiments, the composition further includes one or more emulsion stabilizers (co-emulsifiers), which can be used to stabilize the liquid emulsion upon dilution of the composition into an aqueous solvent. In embodiments, the emulsion stabilizer functions to increase the viscosity of embodiments of the cannabinoid enriched solid composition or the mixture produced by dilution thereof.

Exemplary of an emulsion stabilizer that can be used in the provided compositions is a composition containing a blend of gums, for example, gums used as emulsifying agents, for example, a blend containing one or more of xanthan gum, guar gum and sodium alginate. Exemplary of such an emulsion stabilizer includes the emulsion stabilizer sold under the brand name SALADIZER®, available from TIC Gums, Inc. (Belcamp, Md.). Other gums can be included in the emulsion stabilizer, for example, gum acacia, ester gums and sugar beet pectin. Exemplary emulsion stabilizers include modified food starches. These include the modified gum acacia sold under the name Tic Pretested® Ticamulsion A-2010 Powder, available from TIC Gums, Inc. (Belcamp, Md.). Other exemplary emulsion stabilizers containing an ester gum are, for example, the emulsion stabilizer sold under the name Tic Pretested® Ester Gum 8BG, available from TIC Gums, Inc. (Belcamp, Md.) or Ester Gum 8BG, available from Hercules/Pinova (Brunswick, Ga.). Others sold by Ingredion, Inc (Westchester, Ill.) under the trademarks CAPSUL®, FIRMTEX®, THERMFLO®, THERMTEX®, and TEXTRA® and others, can be included in the compositions provided herein. Other blends of similar gums can also be used as emulsion stabilizers.

The emulsion stabilizer can be added to the water phase, the oil phase, or both the water and the oil phase, during formation of the particulates. In embodiments, the emulsion stabilizer is present in the composition at greater than or equal to about 0.1 wt % or about 0.1% and 1% or about 1%, for example, 0.1%, 0.15%, 0.25%, 0.3%, 0.31%, 0.32%, 0.33%, 0.34%, 0.35%, 0.36%, 0.4%, 0.5%, 0.6%, 0.7%, 0.82%, 0.9% or 1 wt %, or 5%, 10%, 15%, 18%, 20%, or 25%, by weight, or more.

The cannabinoid enriched solid composition may further include one or more flavoring agents, for example, any compound that can add flavor upon dilution into an aqueous liquid. Exemplary of flavors that can be used are fruit flavors, such as guava, kiwi, peach, mango, papaya, pineapple, banana, strawberry, raspberry, blueberry, orange, grapefruit, tangerine, lemon, lime and lemon-lime; cola flavors, tea flavors, coffee flavors, chocolate flavors, dairy flavors, root beer and birch beer flavors, methyl salicylate (wintergreen oil, sweet birch oil), citrus oils and other flavors. Typically, the flavors are safe and/or desirable for human consumption, for example, GRAS or Kosher-certified flavors. An exemplary flavoring agent that can be used in embodiments of the cannabinoid enriched solid composition include lemon oil, for example lemon oil sold by Mission Flavors (Foothill Ranch, Calif.), and D-limonene, for example, 99% GRAS certified D-Limonene, sold by Florida Chemical (Winter Haven, Fla.).

In embodiments, the cannabinoid enriched solid composition further includes one or more pH adjusters which may be added at an appropriate concentration to achieve a desired pH. Suitable pH adjuster are added to adjust the pH of the mixture produced upon dilution of the particulates in water to a pH of greater than or equal to about 2 to less than or equal to about 9, or from about 2 to 8, or 5 to 7.5, or from 3 to 4.0 or 4 to 6. In embodiments, the pH adjuster present in an amount sufficient to adjust the pH of 1 wt % of the composition in deionized water to greater than or equal to about 6 and less than or equal to about 8 at 25° C.

One or more of a plurality of pH adjusting agents can be used. Typically, the pH adjusting agent is safe for human consumption, for example, GRAS certified. The pH adjuster can be citric acid. An exemplary pH adjuster suitable for use with embodiments of the cannabinoid enriched solid composition includes the citric acid sold by Mitsubishi Chemical (Dublin, Ohio). Another exemplary pH adjuster is phosphoric acid, such as Food Grade 80% Phosphoric Acid, sold by Univar. Various buffer compositions may also be employed.

Typically, the concentration of pH adjuster added according to embodiments of the cannabinoid enriched solid composition at less than 5% or about 5%, for example, less than or about 4%, 3.5%, 3%, 2.5%, 2%, 1.5%, 1%, 0.9%, 0.8%, 0.7%, 0.6%, 0.5%, 0.4%, 0.3%, 0.2%, 0.1% or less, by weight, of the cannabinoid enriched solid composition.

In embodiments the cannabinoid enriched solid composition my further include various other components such as soluble fiber. Soluble fibers include any soluble dietary fiber that can be readily fermented in the colon, typically a plant based dietary fiber, for example, a soluble fiber from legumes, vegetables, such as broccoli and carrots, root vegetables, such as potatoes, sweet potatoes and onions, oats, rye, chia, barley and fruits, such as prunes, plums, berries, bananas, apples and pears. Typically, soluble dietary fiber contains non-starch polysaccharides, such as arabinoxylans, cellulose, dextrans, inulin, beta-glucans, fructo-oligosaccharides, oligosaccharides and polysaccharides. Soluble fibers include, but are not limited to, fructo-oligosaccharides, for example, inulins, for example, inulins found in chicory, Jerusalem artichoke, dahlia, garlic, leeks and onions, fructans and water-soluble soybean fiber. Exemplary of a soluble fiber is an inulin, for example, Oliggo-Fiber Instant Inulin (Fibruline® Instant) (supplied by Cosucra-Groupe Warcoing SA, Belgium, sold by Gillco Products, San Marcos, Calif.), containing chicory inulin.

Other additional components include sweeteners, glidents, anti-caking agents, antifoaming agents, and the like.

In one or more embodiments, the cannabinoid enriched solid composition my further include one or more stabilizers, or a stabilizing system. Stabilizers include any compound used to stabilize the cannabinoids and/or other non-polar ingredients in the cannabinoid enriched solid composition, and/or upon dilution of the cannabinoid enriched solid composition in an aqueous solvent. Suitable stabilizer or stabilizing systems include, but are not limited to, carbonates and bicarbonates, acids, antioxidants, and any combination thereof. Typically, the stabilizers or stabilizing system are food-approved, i.e., edible or ingestible, stabilizers, for example, stabilizers that are safe and/or approved for human consumption.

Suitable stabilizers include sodium bicarbonate, potassium bicarbonate, sodium carbonate, potassium carbonate, calcium carbonate, magnesium carbonate, zinc carbonate, and any combination thereof. Other stabilizers include acids such as citric acid, phosphoric acid, adipic acid, ascorbic acid, lactic acid, malic acid, fumaric acid, gluconic acid, succinic acid, tartaric acid, maleic acid, and any combination thereof.

Other stabilizers include antioxidants such as, but are not limited to hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, vitamins and vitamin cofactors, such as vitamin A, vitamin C, vitamin E, vitamin E phosphate and ubiquinone (ubidecarenone, coenzyme Q, coenzyme Q10), ascorbic acid, citric acid, rosemary oil, minerals, such as mineral selenium and manganese, melatonin, alpha-carotene, .beta.-carotene, lycopene, lutein, zeanthin, crypoxanthin, resveratrol, eugenol, quercetin, catechin, gossypol, hesperetin, curcumin, ferulic acid, thymol, hydroxytyrosol, tumeric, thyme, olive oil, lipoic acid, glutathione, gulamine, oxalic acid, tocopherol-derived compounds, di-alpha-tocopheryl phosphate, tocotrienols, butylated hydroxyanisole, butylated hydroxytoluene, ethylenediaminetetraacetic acid, tert-butylhydroquinone, acetic acid, pectin, tocotrienol, tocopherol, coenzyme Q10 (coQ10), zeaxanthin, astaxanthin, canthaxanthin, saponins, limonoids, kaempferol, myricetin, isorhamnetin, proanthocyanidins, quercetin, rutin, luteolin, apigenin, tangeritin, hesperetin, naringenin, eriodictyol, flavan-3-ols (e.g., anthocyanadins), gallocatechins, epicatechin and its gallate forms, epigallocatechin and its gallate forms theaflavin and its gallate forms, thearubigins, isoflavone phytoestrogens, genistein, daidzein, glycitein, anythocyanins, cyaniding, delphinidin, malvidin, pelargonidin and peonidin. In one example, the antioxidant is vitamin C In another example, the antioxidant is a coenzyme Q-containing compounds, such as ubiquinone (ubidecarenone, coenzyme Q, coenzyme Q10).

### Stability

In embodiments, the cannabinoid enriched solid composition or the liquids produced by dilution of the cannabinoid enriched solid composition are free from one or more changes over a period of time, for example, 1 or more days, 1 or more weeks, 1 or more months, or one or more years, for example, 1, 2, 3, 4, 5, 6, 7 or more days, 1, 2, 3, 4, 5, 6, 7, 8, 9, 10 or more weeks, 1, 2, 3, 4, 5, 6, 7, 8, 9, 10, 11, 12 or more months or 1, 2, 3, 4 or more years.

### Oral Bioavailability

In one or more embodiments, the cannabinoid enriched solid composition, or the liquids produced by dilution of the cannabinoid enriched solid composition exhibit a high or relatively high bioavailability, for example, a bioavailability that is higher than a liquid containing the cannabinoid alone (i.e., not formulated according to embodiments disclosed herein). Bioavailability relates to the ability of the body to absorb the cannabinoid into a particular space, tissue cell and/or cellular compartment.

### Embodiments Listing

Accordingly, the disclosure is directed to one or more embodiments and/or aspects including one or more of the following:
E1. A method of treating a medical condition addressed by one or more cannabinoids, comprising:
   providing a feed mixture comprising one or more cannabinoids to an animal capable of producing a foodstuff;
   collecting a cannabinoid enriched foodstuff produced by the animal;
   combining a portion of the cannabinoid enriched foodstuff with an amount of an absorbent, an adsorbent, or a combination thereof, to produce a free-flowing cannabinoid enriched solid composition, and
   providing the cannabinoid enriched solid composition to a recipient in an amount sufficient to treat the medical condition.
E2. The method according to Embodiment E1, wherein the living organism is a bee and the foodstuff is honey.
E3. The method according to Embodiment E1 or E2, wherein the absorbent, the adsorbent, or both, comprise a maltodextrin, a nanoporous starch aerogel, an agglomerated starch comprising a starch binding agent and starch granules, a thermally inhibited agglomerated starch, an amylopectin, a modified waxy maize starch, or a combination thereof.
E4. The method according to any one of Embodiments E1 through E3, wherein the feed mixture comprises one or more tetrahydrocannabinols, cannabidiol, or a combination thereof.
E5. The method according to any one of Embodiments E1 through E4, wherein the feed mixture comprises from about 0.1 wt % to about 5 wt % of the one or more cannabinoids, based on the total amount of the feed mixture present.
E6. The method according to any one of Embodiments E1 through E5, wherein a concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the composition present.
E7. The method according to any one of Embodiments E1 through E6, wherein the cannabinoid is present in the cannabinoid enriched solid composition as a water soluble conjugate, a water dispersible conjugate, or a combination thereof.
E8. The method according to any one of Embodiments E1 through E7, wherein the cannabinoid is present in the cannabinoid enriched solid composition as a glycoside, as a glutathione conjugate, or a combination thereof.
E9. The method according to any one of Embodiments E1 through E8, wherein the cannabinoid enriched solid composition comprises from about 10 wt % to about 90 wt % of the cannabinoid enriched foodstuff, preferably from about 30 wt % to about 60 wt %, based on the total amount of the composition present.
E10. A cannabinoid enriched composition, comprising:
   a cannabinoid enriched foodstuff produced by, or comprising a portion of an organism in the animal kingdom which has been fed with a feed comprising one or more cannabinoids.
E11. The cannabinoid enriched composition according to Embodiment E10, wherein the organism in the animal kingdom is an insect.
E12. The cannabinoid enriched composition according to Embodiment E10 or E11, wherein the cannabinoid enriched foodstuff further comprises an absorbent, an adsorbent, or a combination thereof, present in an amount such that the composition is a free-flowing solid.
E13. A cannabinoid enriched composition, comprising:
   a cannabinoid enriched foodstuff produced by a living organism in the animal kingdom; in combination with an absorbent, an adsorbent, or a combination thereof, present in an amount such that the composition is a free-flowing solid.
E14. The cannabinoid enriched composition according to any one of Embodiments E10 through E13, wherein the cannabinoid enriched foodstuff is honey produced by bees which have been fed a feed mixture comprising one or more cannabinoids.
E15. The cannabinoid enriched composition according to any one of Embodiments E10 through E14, wherein the feed mixture comprises one or more tetrahydrocannabinols, cannabidiol, or a combination thereof.
E16. The cannabinoid enriched composition according to any one of Embodiments E10 through E15, wherein the feed mixture comprises from about 0.1 wt % to about 5 wt % of the one or more cannabinoids, based on the total amount of the feed mixture present.
E17. The cannabinoid enriched composition according to any one of Embodiments E10 through E16, wherein the absorbent, the adsorbent, or both, comprise a maltodextrin, a nanoporous starch aerogel, an agglomerated starch comprising a starch binding agent and starch granules, a thermally inhibited agglomerated starch, an amylopectin, a modified waxy maize starch, or a combination thereof.
E18. The cannabinoid enriched composition according to any one of Embodiments E10 through E17, wherein a concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the cannabinoid enriched composition present.
E19. The cannabinoid enriched composition according to any one of Embodiments E10 through E18, wherein the cannabinoid is present in the cannabinoid enriched composition as a water soluble conjugate a water dispersible conjugate, or a combination thereof.
E20. The cannabinoid enriched composition according to any one of Embodiments E10 through E19, wherein the cannabinoid is present in the cannabinoid enriched solid composition as a glycoside, as a glutathione conjugate, or a combination thereof.
E21. The cannabinoid enriched composition according to any one of Embodiments E10 through E20 comprising from about 10 wt % to about 90 wt % of the cannabinoid enriched foodstuff, preferably from about 30 wt % to about 60 wt %, based on the total amount of the composition present.
E22. A cannabinoid enriched composition, comprising:
   a cannabinoid enriched foodstuff produced by feeding a living organism in the animal kingdom a feed comprising one or more cannabinoids; harvesting at least a portion of the animal to produce the cannabinoid enriched foodstuff.
E23. The cannabinoid enriched composition according to Embodiment E22, wherein the living organism in the animal kingdom is an insect, preferably a cricket, a grasshopper, and/or a moth.

### Examples

The foregoing discussion can be further described with reference to the following non-limiting examples.

A sample of cannabinoid enriched honey (the “foodstuff”) was obtained from Dionysus Management Co. Data from the certificate of analysis of the honey is shown in Table 1 below.

TABLE 1

| | LOD | LOQ | Result | Result |
|---|---|---|---|---|
| Analyte | mg/g | mg/g | Wt % | mg/g |
| THCa | 0.00 | 0.00 | ND | ND |
| Δ9-THC | 0.00 | 0.00 | 1.117 | 0.1117 |
| Δ8-THC | 0.00 | 0.00 | ND | ND |
| CBDa | 0.00 | 0.00 | ND | ND |
| CBD | 0.00 | 0.00 | 0.009 | 0.0009 |
| CBDV | 0.00 | 0.00 | ND | ND |
| CBN | 0.00 | 0.00 | 0.012 | 0.0012 |
| CBGa | 0.00 | 0.00 | ND | ND |
| CBG | 0.00 | 0.00 | 0.028 | 0.0028 |
| CBC | 0.00 | 0.00 | 0.012 | 0.0012 |
| Total THC | | | 1.177 | 0.1177 |

A 1:1 wt/wt mixture of the foodstuff was mixed with N-Zorbit 2144 plating agent by slowly adding the honey to the absorbent while mixing in a blender in the presence of solid carbon dioxide (dry ice) to obtain embrittlement of the particles and the mixture was ground using commercial grinding equipment to reduce particle size, remove agglomerates, and to achieve a uniform mixture in the form of a dry, free-flowing powder composition. Additional mixing was done utilizing the foodstuff and the absorbent. Free-flowing powder compositions were obtained at foodstuff levels from about 10 wt % to about 60 wt %, with 40 wt % being optimal.

A 1 wt % solution of this cannabinoid enriched solid composition was dissolved in water and found to have significant biological effect on humans conducive to treatment of anxiety and other disorders known to be treatable by cannabinoids.

The cannabinoid enriched solid composition, a 1 wt % aqueous solution of the cannabinoid enriched solid composition, and a 1 wt % aqueous mixture of the cannabinoid enriched solid composition in fruit juice were prepared and analyzed via HPLC-2dUV for free cannabinoids. The chromatograms are found in FIGS. 1, 2, and 3. As these data show, despite the pronounce biological effect this composition possesses, analysis shows only trace concentrations of the cannabinoids that were present in the feed that was fed to the bees.

### Insect Derived Foodstuff

Commercially available crickets were feed a combination of cricket feed and cannabis over an 11 week period until the crickets obtained an average size of about 20 mm One week prior to harvesting, all cannabis present in the feed was removed to ensure no undigested cannabis would be present. The crickets we dried and ground into power according to standard industry practice. The cricket powder was found to have a pronounced biological effect on volunteer human test subjects. The powder was assayed for cannabinols via standard HPLC methods. The chromatogram of this analysis is shown in FIG. 4. The results are presented in Table 2 below:

TABLE 2

| | LOD | LOQ | Result | Result |
|---|---|---|---|---|
| Analyte | mg/g | mg/g | Wt % | mg/g |
| THCa | 0.00 | 0.00 | 0.087 | 0.87 |
| Δ9-THC | 0.00 | 0.00 | 0.033 | 0.33 |
| Δ8-THC | 0.00 | 0.00 | 0.011 | 0.11 |
| CBDa | 0.00 | 0.00 | ND | ND |
| CBD | 0.00 | 0.00 | 0.016 | 0.16 |
| CBN | 0.00 | 0.00 | 0.003 | 0.03 |
| CBGa | 0.00 | 0.00 | ND | ND |
| CBG | 0.00 | 0.00 | 0.056 | 0.56 |
| CBC | 0.00 | 0.00 | ND | ND |
| Total THC | | | 0.109 | 1.09 |

These data suggest that cannabinoids may be expressed as cardiac glycosides and/or other water soluble metabolites, conjugates, derivatives and/or the like produced by the insects, which may be utilized to produce a foodstuff comprising cannabinoids according to various aspects of the disclosure.

All documents described herein are incorporated by reference herein, including any priority documents and/or testing procedures to the extent they are not inconsistent with this text. As is apparent from the foregoing general description and the specific embodiments, and aspects of the disclosure have been illustrated and described, various modifications can be made without departing from the spirit and scope of the disclosure. Accordingly, it is not intended that the disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

1. A method of treating a medical condition addressed by one or more cannabinoids, comprising:
providing a feed mixture comprising one or more cannabinoids to an animal capable of producing a foodstuff;
harvesting a cannabinoid enriched foodstuff produced by the animal;
combining a portion of the cannabinoid enriched foodstuff with an amount of an absorbent, an adsorbent, or a combination thereof, to produce a free-flowing cannabinoid enriched solid composition, and
providing the cannabinoid enriched solid composition to a recipient in an amount sufficient to treat the medical condition.

2. The method of claim 1, wherein the living organism is a bee and the foodstuff is honey.

3. The method of claim 1, wherein the absorbent, the adsorbent, or both, comprise a maltodextrin, a nanoporous starch aerogel, an agglomerated starch comprising a starch binding agent and starch granules, a thermally inhibited agglomerated starch, an amylopectin, a modified waxy maize starch, or a combination thereof.

4. The method of claim 1, wherein the feed mixture comprises one or more tetrahydrocannabinols, cannabidiol, or a combination thereof.

5. The method of claim 1, wherein the feed mixture comprises from about 0.1 wt % to about 5 wt % of the one or more cannabinoids, based on the total amount of the feed mixture present.

6. The method of claim 1, wherein a concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the composition present.

7. The method of claim 1, wherein the cannabinoid is present in the cannabinoid enriched solid composition as a water soluble conjugate, a water dispersible conjugate, or a combination thereof.

8. The method of claim 1, wherein the cannabinoid is present in the cannabinoid enriched solid composition as a glycoside, as a glutathione conjugate, or a combination thereof.

9. The method of claim 1, wherein the cannabinoid enriched solid composition comprises from about 10 wt % to about 90 wt % of the cannabinoid enriched foodstuff, preferably from about 30 wt % to about 60 wt %, based on the total amount of the composition present.

10. A cannabinoid enriched composition, comprising:
a cannabinoid enriched foodstuff produced by, or comprising a portion of an organism in the animal kingdom which has been fed with a feed comprising one or more cannabinoids.

11. The cannabinoid enriched composition of claim 10, wherein the organism in the animal kingdom is an insect.

12. The cannabinoid enriched composition of claim 10, wherein the cannabinoid enriched foodstuff further comprises an absorbent, an adsorbent, or a combination thereof, present in an amount such that the composition is a free-flowing solid.

13. The cannabinoid enriched composition of claim 12, wherein the cannabinoid enriched foodstuff is honey produced by bees which have been fed a feed mixture comprising one or more cannabinoids.

14. The composition of claim 12, wherein the absorbent, the adsorbent, or both, comprise a maltodextrin, a nanoporous starch aerogel, an agglomerated starch comprising a starch binding agent and starch granules, a thermally inhibited agglomerated starch, an amylopectin, a modified waxy maize starch, or a combination thereof.

15. The composition of claim 10, wherein the feed mixture comprises from about 0.1 wt % to about 5 wt % of the one or more cannabinoids, based on the total amount of the feed mixture present.

16. The composition of claim 10, wherein a concentration of free THC, CBD, or both in the cannabinoid enriched composition is less than or equal to about 1000 ppm, preferably less than or equal to about 500 ppm, preferably less than or equal to about 100 ppm, preferably less than or equal to about 50 ppm, preferably less than or equal to about 20 ppm, based on the total amount of the cannabinoid enriched composition present.

17. The composition of claim 10, wherein at least one cannabinoid is present in the cannabinoid enriched composition as a water soluble conjugate, a water dispersible conjugate, or a combination thereof.

18. The composition of claim 10, wherein the cannabinoid is present in the cannabinoid enriched solid composition as a glycoside, as a glutathione conjugate, or a combination thereof.

19. The composition of claim 10, comprising from about 10 wt % to about 90 wt % of the cannabinoid enriched foodstuff, preferably from about 30 wt % to about 60 wt %, based on the total amount of the composition present.

20. The composition of claim 10, wherein the feed mixture comprises one or more tetrahydrocannabinols, cannabidiol, or a combination thereof.
